(19) United States
(12) Patent Application Publication
(10) Pub. No.: US 2009/0214600 A1
(43) Pub. Date: Aug. 27, 2009

(54) METHODS AND COMPOSITIONS FOR GASTRIC RESISTANT ORAL FORMULATIONS FOR INTESTINAL DELIVERY

(76) Inventor: DANIEL R. DEBROUSE, Oklahoma City, OK (US)

Correspondence Address: NYEMASTER, GOODE, WEST, HANSELL & O'BRIEN, P.C., 700 Walnut Street, Suite 1600, Des Moines, IA, 50309-3899, US

(21) Appl. No.: 12/036,867

(22) Filed: Feb. 25, 2008

(51) Int. Cl. A61K 9/00

(52) U.S. Cl. 424/400

(57) ABSTRACT

An oral formulation for mammalian administration and treatment utilizing bioactive pharmaceuticals in need of gastric resistance to promote intestinal absorption. The oral formulation utilizes lower dosages to achieve higher plasma levels and delivers permeable solubilized bioactive pharmaceuticals directly to intestinal mucosa, avoiding premature gastric disintegration. Further, it delivers water and lipid soluble bioactives directly to the intestines overcoming the molecular characteristics previously limiting the therapeutic administration. Upon oral ingestion of the formulation, a disaccharide component is hydrolyzed by intestinal enzymes to bring the bioactive pharmaceutical in contact with the surface of intestinal mucosa. A method of administering oral bioactive pharmaceuticals as well as a method for manufacturing the formulation is disclosed.

### FIELD OF THE INVENTION

The invention relates to oral dosage formulations that resist gastric disintegration and are absorbed in the intestines is disclosed, in addition to a method of administering oral bioactive pharmaceuticals and methods for manufacturing the oral formulation.

### BACKGROUND OF THE INVENTION

Upon mammalian administration of a bioactive pharmaceutical, the biologically active product must transverse several semi-permeable cellular membrane barriers before reaching the systemic circulation. A limitation to a bioactive pharmaceutical's absorption is that such membranes generally exhibit a high degree of selectivity to the passage of drug molecules and are composed primarily of a phospholipid bi-layer. The lipid network, in addition to providing stability to the membrane, displays globular proteins of various size and composition which are involved in processes ranging from nutrient absorption, cellular regulation, and the transport of molecules across the lipid bi-layer. In general, bioactive pharmaceuticals may transverse this membrane barrier by any one or combination of the following mechanisms: passive diffusion, facilitated passive diffusion, active transport and pinocytosis.

Passive diffusion is a transport process across the bi-layer that is dependent upon the concentration gradient of the solute. For example, drug molecules able to traverse the bi-layer by this mechanism are rapidly removed by the systemic circulation due to a high concentration at the site of dissolution and an initially low blood concentration, setting up a large difference in gradient. The prerequisites to simple diffusion are the lipid solubility of a drug, its degree of ionization, molecular weight, and the area of the absorptive surface.

Facilitated passive diffusion is a process driven by a carrier protein component that reversibly combines with a drug molecule at the cell exterior membrane and rapidly transports it across the bi-layer releasing the drug molecule at the interior surface. Carrier-mediated diffusion of this type is characterized by selectivity and saturability. This mode of transport is relatively rare in conventional drug administration and is limited by both the drug molecule's molecular configuration and the availability of protein carriers.

The active transport process is also characterized by selectivity and saturability. However with active transport, energy expenditure is required. Therefore, active transport is severely limited to endogenous substances. Finally, drug absorption pinocytosis is presumed to play a very minimal role and occurs simply by the chance presence of a drug molecule in the right place at the right time. In this process the cell membrane invaginates enclosing fluid and digestive particles, forming a vesicle that later detaches and moves to the cell interior. Pinocytosis may be exploited to play a major absorptive role with protein drug molecules.

Oral routes of administration are generally preferred over non-oral routes of administration, such as IV, IM, and rectal to name a few, for a variety of patient preference, cost, formulation, storage and stability reasons. Patients able to ingest oral-formulations prefer such oral formulation as an easier option for patients to comply with outside an inpatient setting. Oral formulations are generally the least costly formulation and require minimal safety concerns associated with handling, storage and disposal. Oral formulations are also preferred in many instances as they often provide a delivery method that mimics the physiological path of secretion or digestion of various compounds. Additionally, the ideal onset of action of various bioactive pharmaceuticals is achieved via oral administration.

A well-known example of a bioactive pharmaceutical in need of an oral formulation is Ceftriaxone sodium. Ceftriaxone sodium is a broad spectrum third-generation cephalosporin antibiotic for which conventional molecular science has failed to formulate an oral delivery system. Rather, effective bacterialcidal therapy relies upon either intravenous (IV) infusion or intramuscular (IM) injection. Since the U.S. Food and Drug Administration first approved this antibiotic in 1985, physicians have successfully treated more than 100 million patients worldwide with infectious processes, utilizing the IV and IM routes of administration.

Ceftriaxone sodium is a semi-synthetic cephalosporin. The molecule presents as a white to yellowish-orange crystalline powder at 25 degrees Celsius, having the following chemical formula, C₁₈H₁₆N₈Na₂O₇S₃. Ceftriaxone is readily soluble in water, only sparingly to slightly soluble in methanol and ethanol respectively, and is relatively impermeable to the lipid fraction of cellular membranes. Ceftriaxone's impermeability is a result of its ionization and its degree of electrical resistance. An oral Ceftriaxone sodium formulation would beneficially reduce conventional burdens upon both patients and health care professionals.

The oral formulation allows traditional IV, IM or other non-orally administered therapies to be consistently and effectively administered in an oral route. For example, Ceftriaxone therapy has previously been entirely dependent upon IV infusion or IM administration due to various molecular characteristics. There are at least three molecular characteristics contributing to Ceftriaxone's limited therapeutic administration practices. First, the molecule displays a high degree of polarity. Although many polar molecules are know to transverse the mucosa lipid bi-layer by passive transport through specialized pores, Ceftriaxone absorption by this mechanism is remarkably poor. Second, Ceftriaxone is an ionized molecule, and as such is resistant to lipid dissolution, establishing an immediate barrier to the necessary lipid diffusion of Ceftriaxone across the mucosa bi-layer. Protein gated ion channels are well known components of the lipid bi-layer, however, passage of molecules through such channels is usually restricted to inorganic ions much smaller than the organic Ceftriaxone molecule. Third, the Ceftriaxone molecule presents a high degree of electrical resistance to the lipid bi-layer due to its level of polarity.

These barriers to lipid bi-layer traversion are not always sufficient to prevent diffusion of biologically active agents. If these barriers were absolute, numerous biologically active agents commonly used would have absolutely no therapeutic value. For Ceftriaxone it is clear that drug absorption is also mediated by its concentration gradient. In the case of charged molecules, the electrical potential difference across the cellular membrane provides Ceftriaxone with sufficient absorption to combat bacterial invasion with IV or IM administration.

The following tables show the average Ceftriaxone plasma concentrations after single dose IV administration (Table 1) and single dose IM administration (Table 2).

TABLE 1
Plasma Concentrations (ug/mL)

| Dose | 0.5 hr | 1 hr | 2 hr | 4 hr | 6 hr | 8 hr | 12 hr | 16 hr | 24 hr |
|---|---|---|---|---|---|---|---|---|---|
| 1.0 g | 151 | 111 | 88 | 67 | 53 | 43 | 28 | 18 | 9 |

TABLE 2
Plasma Concentrations (ug/mL) (ND = Not Detected)

| Dose | 0.5 hr | 1 hr | 2 hr | 4 hr | 6 hr | 8 hr | 12 hr | 16 hr | 24 hr |
|---|---|---|---|---|---|---|---|---|---|
| 1.0 g | 40 | 68 | 76 | 68 | 56 | 44 | 29 | ND | ND |

Metabolism studies of Ceftriaxone show that approximately 33-67% of a dose is excreted as unchanged drug in the urine. The remainder of a Ceftriaxone dose is secreted in bile and found microbiologically inactive in the feces. The excretion processes show the antibiotic's poor absorbability and that higher plasma concentrations, with lower dosages, are achievable at the intestinal mucosa. As a result, the oral formulation permits higher blood Ceftriaxone levels through utilizing lower dosages. However, oral Ceftriaxone administration is entirely dependent upon the oral formulation's ability to deliver permeable solubilized antibiotic directly to the intestinal mucosa, bypassing any premature disintegration in gastric fluids.

Modification of Ceftriaxone's impermeability characteristics results in the small intestine being the ideal target for Ceftriaxone dissolution. Digestion is not always considered a significant process in drug absorption. However, digestion becomes a critical process with the novel oral formulation of Ceftriaxone due to the exterior coating utilized. Mechanical digestion is initiated upon the swallowing of the oral formulation. Swallowing may be considered to typically consist of three steps: movement of the formulation through the mouth into the pharynx; through the pharynx into the esophagus; and finally through the esophagus and into the stomach. In the stomach, oral Ceftriaxone must retain both its physical and chemical characteristics remaining relatively unchanged until passage into the duodenum. If the oral formulation were to disintegrate prematurely upon entering the stomach, absorbability of unsolubilized Ceftriaxone would be very poor, yielding plasma concentrations much smaller than those obtained as a result of IV or IM administration.

Chemical digestion proceeds upon the oral formulation's entry into the jejunum region of the small intestine. The disaccharide component of the oral dosage formulation is hydrolyzed by intestinal enzymes, such as sucrase, lactase, and maltase, thereby releasing the solubilized Ceftriaxone. Mechanical digestion again plays a significant role by inducing intestinal churning to bring the lipid-solubilized Ceftriaxone, or any lipid-soluble or water-soluble bioactive pharmaceutical, in contact with the surface of intestinal mucosa. In yet another aspect of the present invention, once the oral formulation has been hydrolyzed and the bioactive is available for intestinal absorption, cleavage of the bioactive molecule is yet another barrier to absorption that must be addressed. For example, partial cleavage of the Ceftriaxone molecule is catalyzed by intestinal fluid enzymes. Therefore another chemical mechanism involving enzymatic inhibition in the local intestinal region of formulation disintegration impacts Ceftriaxone delivery across intestinal mucosa.

The jejunum region of the small intestine is the ideal region for the oral formulation disintegration and thus release of the bioactive pharmaceutical contained therein for two primary reasons. First, the small intestine is where the vast majority of digestion and subsequent absorption of digestive end products occurs and, second, the small intestine maintains a large surface area prone to absorption, greatly increasing the probability of drug diffusion by one or any combination of at least four mechanisms. Premature disintegration in the stomach would expose the bioactive pharmaceutical to a generally degradative environment, most often resulting in an inadequate absorption of antibiotic resulting in an inadequate therapeutic value. Likewise the formulation's disintegration within the large intestine would result in the excretion of dose majority as waste, as is the primary function of the large intestine.

Developing an oral Ceftriaxone formulation has been an unattainable task, illustrative of the need for an oral formulation for mammalian administration of Ceftriaxone. It is also illustrative of a class of bioactive pharmaceuticals in need of formulations to allow those drug products, previously available in only IV or other non-oral routes of administration, to be re-formulated to promote and allow intestinal absorption to achieve blood plasma concentrations at least equal to if not superior to those obtained after oral administration. Numerous antibiotics, chemotherapeutics, other bioactive pharmaceuticals, neutraceuticals, genetic products, vitamins and minerals, to name a few, are limited in their routes of administration due to the various molecular properties previously precluding any effective oral administration.

Therefore it is a primary object feature or advantage of the present invention to improve over the state of the art to provide a formulation for oral mammalian administration of lipid- and water-soluble bioactive pharmaceuticals previously incapable of oral administration.

A further object, feature, or advantage of the invention is to provide a method for treatment of medical conditions with a novel oral formulation for lipid- and water-soluble bioactive pharmaceuticals.

A further object, feature, or advantage of the invention is to provide an oral formulation for capable for bioactive pharmaceuticals that can be used in medical treatment applications.

A further object, feature, or advantage of the invention is to provide an oral formulation resistant to gastric disintegration but readily dissolvable in the intestinal pool.

Another object, feature, or advantage of the invention is to provide a method of formulating bioactive pharmaceuticals to provide for oral administration to achieve higher bioactive plasma concentrations through the use of lower dosages in comparison to existing IV or IM dosage formulations.

One or more of these and/or other objects, features, or advantages of the present invention will become apparent from the specification and claims that follow.

### BRIEF SUMMARY OF THE INVENTION

The present invention includes a novel oral formulation for both water-soluble and lipid-soluble bioactive pharmaceuticals that are resistant to gastric disintegration, providing an intestinal breeching delivery system. The delivery system is readily dissolvable in the intestinal pool and capable of transversing intestinal cellular membrane barriers. The delivery system brings the bioactive pharmaceuticals directly to the jejunum region of the small intestines where the formulation disintegrates upon contact with digestive enzymes. Upon disintegration, the biologically active pharmaceutical is released in solubilized form and can be readily absorbed. This is all accomplished in a system that uses an inner core containing the bioactive pharmaceutical and a protectant outer core membrane.

### BRIEF DESCRIPTION OF THE DRAWINGS

FIG. 1 shows the amide linkage in a Ceftriaxone molecule.

FIG. 2 shows the Ceftriaxone molecule containing two identified amino acid-like residues.

FIG. 3 shows a schematic model of a sodium-dependent amino acid transporter.

FIG. 4 shows Ceftriaxone coupled with a fatty acid, such as linoleic acid.

FIG. 5 shows two distinct functional regions of the Ceftriaxone sodium oral formulation.

FIG. 6 shows the time- or controlled- or extended-release formulation showing two layers of bioactive pharmaceutical dosing as one embodiment of the present invention's oral formulation.

### DETAILED DESCRIPTION OF THE INVENTION

Unless defined otherwise, all technical and scientific terms used herein have the same meaning as commonly understood by one of ordinary skill in the art to which this invention belongs. Unless mentioned otherwise, the techniques employed or contemplated herein are standard methodologies well known to one of ordinary skill in the art. The materials, processes and examples described in the description of the invention are illustrative only and not intended to be limiting to the scope of the invention in any manner. Many modifications and other embodiments of the inventions set forth herein will come to mind to one skilled in the art to which these inventions pertain having the benefit of the teachings presented in the foregoing descriptions and the associated drawings. Therefore, it is to be understood that the inventions are not to be limited to the specific embodiments disclosed and that modifications and other embodiments are intended to be included within the scope of the appended claims.

For purposes of this application the following terms, as used herein, shall have the definitions recited herein. However, the terms defined below are more fully defined by reference to the specification as a whole.

The term “bioactive” or “bioactive pharmaceutical” includes any molecule, compound or drug interacting with and/or having effects on any cell tissue in a human being or any mammal. The definition is not limiting with regard to the chemical structure, active ingredient and/or component, or biological activity and/or toxicity. This includes, but is not limited to, any compound or drug that activates, causes activation, inhibits, causes inhibition, both competitive and non-competitive agonists and antagonists, prodrugs, and analogs. For exemplary purposes only, a bioactive pharmaceutical may include pharmaceuticals, including any water or lipid soluble pharmacologically active agent (such as, but not limited to, antibiotics, antivirals, anti-inflammatory agents, chemotherapies, vaccines, peptides, peptide mimics, insulin, DNA, RNA, and carotenoids), neutraceuticals, vitamins, minerals, and combinations of the same.

The term “pharmaceutical equivalents” includes, without limitation, pharmaceutically acceptable salts, hydrates, metabolites, and prodrugs. They may also include equivalents for the formulation components other than the bioactive pharmaceutical, including without limitation, an acceptable carrier, diluent, excipient, wetting agent, buffering agent, suspending agent, lubricating agent, adjuvant, vehicle, delivery system, emulsifier, disintegrant, absorbent, preservative, surfactant, colorant, flavorant, sweetener, or other components that would be suitable for use in a pharmaceutical composition. Many pharmaceutically acceptable equivalents are expected to have the same or similar in vitro or in vivo activity as the compounds of the invention.

The terms “therapeutically effective” or “pharmaceutically effective” shall mean an amount of each active component of the pharmaceutical composition (i.e., bioactive pharmaceutical, bioactive, drug, drug molecule, etc.) or method that is sufficient to show a meaningful patient benefit (i.e., treatment, prevention, amelioration, or a decrease in the frequency of the condition or symptom being treated), as determined by the methods and protocols disclosed herein. When applied to an individual active ingredient, administered alone, the term refers to that ingredient alone. When applied to a combination, the term refers to combined amounts of the active ingredients that result in the therapeutic effect, whether administered in combination, serially or simultaneously.

The terms “transverse” or “translocate” refers to the term known to those of ordinary skill in the art, including the specific mechanism of crossing a lipid bi-layer.

The formulation of the present invention is suited for mammalian oral administration of water- and lipid-soluble bioactive pharmaceuticals. An example of a bioactive pharmaceutical capable of use in the present invention's oral formulation is Ceftriaxone, a preferred drug active for use herein. The oral formulation consists of all natural components that are widely distributed as food and drug additives. Advantageously, the present invention is especially useful for oral administration to human patients, to replace alternative IV and IM routes of administration previously used, due to the bioactive's molecular and pharmalogical characteristics previously precluding oral administration.

The present invention provides an oral formulation for bioactive pharmaceuticals, both lipid and water soluble, resistant to gastric disintegration and composed of a novel intestinal breeching delivery system capable of transversing intestinal cellular membrane barriers. The oral formulation for the drug delivery system of the present invention may be used for the intestinal delivery of bioactive pharmaceuticals previously limited to or including non-oral formulations. These include but are not limited to any water or lipid soluble pharmacologically active agent (such as antibiotics, antivirals, anti-inflammatory agents, chemotherapies, vaccines, peptides, peptide mimics, insulin, DNA, RNA, and carotenoids), neutraceuticals, vitamins, minerals, and combinations of the same.

The formulation according to the invention allows for oral formulations that are resistant to gastric disintegration but readily dissolvable in the intestinal pool. A bioactive pharmaceutical is formulated within a novel intestinal breeching drug delivery system resistant to gastric disintegration. The formulation is designed to deliver both water- and lipid-soluble bioactives directly to the jejunum region of the small intestine. Once excreted from the duodenum, the formulation readily disintegrates upon contact with digestive enzymes serving to release its solubilized internal core of pharmaceutically active agent or agents.

The formulation according to the present invention overcomes the enzymatic breakdown by gastric acids by the formulation's two regions of distinct function, an inner core region and a protective outer membrane region. The inner core region consists of a lipid solubilized bioactive (drug active bioactive pharmaceutical) held in conjunction with specific targeting molecules for the active transport receptor proteins lining the surface of intestinal cells. The targeting molecules promote the active transport of the bioactive pharmaceutical across the lipid bi-layer where it may be processed and absorbed into the blood and lymph. In addition to targeting molecules, the inner core region contains both protectant molecules, which establish a barrier between the bioactive and the digestive pool until the core collision with the intestinal surface, and a lipid solubilizing base medium, which separates the core region from the intestinal digestible outer region and aids in bi-layer dissolution of the core components.

The formulation of the present invention is designed to promote absorption through various mechanisms, including for example, simple diffusion and carrier-mediated diffusion. In order to promote such forms of absorption, the formulation must first be gastric resistant. The formulation of the bioactive in the inner core protects its molecular structure from the harsh conditions of the gastric cavity and promotes its exposure only to intestinal enzymes. Special targeting molecules are also contained in the inner core of the formulation to promote carrier-mediated diffusion of the bioactive.

The prerequisites to simple diffusion are the lipid solubility of a drug, its degree of ionization, molecular weight, and the area of the absorptive surface. One inner core function of the oral formulation is to provide either a water- or lipid-solubilized droplet optimizing its potential chances of transversion by simple diffusion. For example, a lipid-solubilized droplet of Ceftriaxone is weakly attracted to the core lipid base components and may rapidly be dispersed at the membrane surface, establishing a contacted difference in the concentration gradient between the gastrointestinal fluid and the blood.

Carrier-mediated diffusion is the second and very significant target of oral absorption. Special targeting molecules are present within the inner core of the oral formulation to assist in this absorption pathway. These interact with a specific type of carrier protein abundantly distributed over the surface of the exterior membrane. This mono-targeting mechanism results in sufficient bioactive absorption and provides appropriate blood concentrations for successful therapy. However, two types of targeting molecules are weakly conjuncted within the inner core. One type carries the bioactive to the connective tissue between intestinal cells where it may then be transported in complex by an abundant carrier operating within this region. Alternatively, active transport is the second type component to oral absorption of the bioactive.

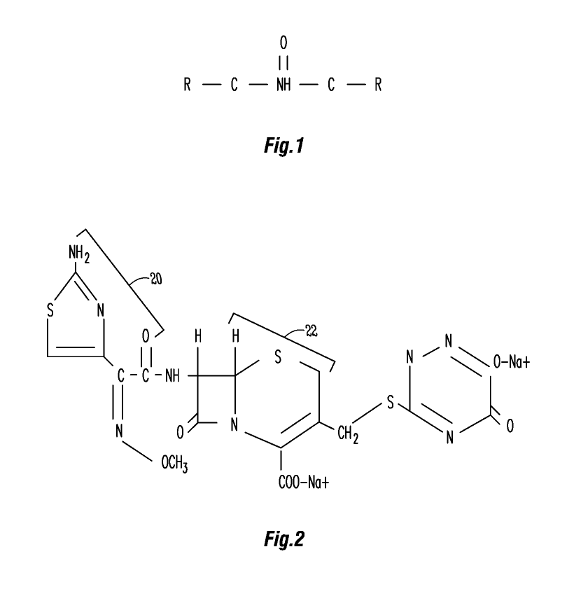

Various molecular limitations must be overcome by the present formulation in order to promote absorption through simple diffusion and carrier-mediated diffusion. For example, Ceftriaxone's limited degree of intestinal mucosal absorption is at least in part due to the molecule's polarity, degree of ionization and its electrical resistance to the phospholipid bi-layer. Additionally, a further inhibitory process is of critical concern. Ceftriaxone's molecular structure shows at least one site at risk of enzymatic cleavage within the intestinal fluid. FIG. 1 shows the amide linkage contained in a portion of a Ceftriaxone molecule, the location of possible protease cleavage within the intestinal fluid. The amide linkage joining the traditional cephalosporin unit to one of the Ceftriaxone specific side chains, closely resembles and thus possesses the characteristics of a peptide linkage. As a result, the Ceftriaxone molecule is susceptible to protease cleavage within the intestinal fluid and plasma. Cleavage of Ceftriaxone within the plasma is one explanation for increased administrative doses required to achieve a therapeutic value. Therefore, the enhancement and translocation mechanisms of the present invention address the cleavage issues for Ceftriaxone and other molecules with the same limitation, through protective measures upon formulation disintegration and subsequent bioactive release. The present invention contemplates at least two separate protective mechanisms for the bioactive's translocation across the intestinal mucosa, each corresponding to a unique formulation process.

The first protective mechanism of a bioactive's translocation across the intestinal mucosa is mediated by sodium-dependent amino acid transportation. FIG. 2 shows Ceftriaxone's molecular structure, including two sites of amino acid “residue” 20, 22 resemblances. Under optimal luminal conditions, Ceftriaxone may be mistaken for an amino acid di- or tri-peptide and transported intracellularly by transmembrane carriers involved in the uptake of such solutes. For example, at least four main types of sodium-dependent amino acid transporters are known to be present within the luminal plasma membrane of the absorptive cells. These transporters may bind amino acid solutes only after first binding a molecule of sodium.

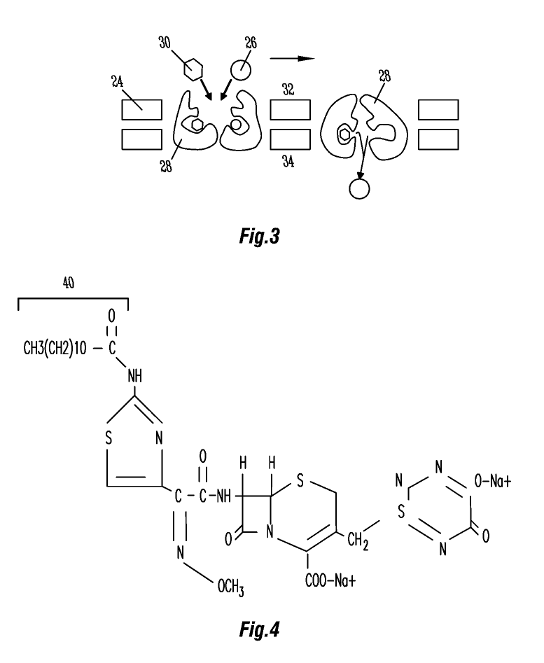

Sodium-dependent amino acid carrier transport is thought to involve the transfer of solute across the lipid bi-layer by undergoing reversible conformational changes that alternately expose the solute binding sites first on one side of the membrane and then on the other. The interaction of solute with carrier resembles an enzyme-substrate reaction. Furthermore, the carrier may have one or more specific binding sites for its solute type that are exposed only after the carrier's initial binding of the sodium ion. FIG. 3 shows a schematic model of a sodium-dependent amino acid transporter along an absorptive membrane 24. The transport of amino acid is dependent upon initial binding of sodium ion 26 occurring through a symport process, facilitated by a symport carrier protein 28 along an absorptive membrane 24 permitting absorption 34.

Ceftriaxone uptake, like many other bioactive pharmaceuticals, at least in part, involves the active transport of the drug molecule by the type of sodium-dependent amino acid 30 translocation process described above, utilizing transport proteins within the luminal plasma membrane of absorptive cells. Some dose portion of Ceftriaxone is absorbed after oral administration of an enterically coated formulation, suggesting that Ceftriaxone translocation involves, at least in part, the sodium-dependent amino acid carrier transport. Dose absorption is variable, in part, due to its dependence upon intestinal lumen sodium concentrations in the apical fluid 32 surrounding the absorptive membrane. Intestinal fluid sodium concentrations vary in patients, for example, depending upon dietary intake at, prior to and after dose administration. In addition to the sodium-dependent translocation process, a calcium-dependent translocation mechanism is also available for a bioactive's uptake at the apical membrane, operating with the equivalent mechanism of a calcium-dependent amino acid carrier transport.

Various bioactive formulation processes have been identified to utilize the intestinal mucosal translocation process. A non-traditional “naked encapsulation” requires formulation of the bioactive molecule within a delivery system specifically targeting intestinal lumen formulation disintegration. This system is absent of modulating and enhancing components to simply deliver gastrically protected bioactive directly to the small intestine.

Alternatively, an enhancer-mediated “naked encapsulation” involves the intestinal delivery of a protected bioactive with sodium chloride incorporated as a translocation enhancement factor, according to the bioactive's dose. The sodium chloride enhancer is added at a specific mole ratio sufficient to trigger sodium-dependent amino acid translocation of the bioactive molecule. This naked encapsulation process increases the bioactive's bioavailability, resulting in more stable, less variable dose absorption at the mucosal membrane.

A further alternative process is an enhancer-mediated assisted-modulator “naked encapsulation” involving dose delivery to the apical surface of the intestinal mucosa. In addition to a sodium chloride enhancer, a pH modulator is incorporated into the oral dose to inhibit protease activity within the intestinal fluid immediately surrounding the region of formulation disintegration, to inhibit the “peptide” linkage within a Ceftriaxone molecule, or another target linkage within any other bioactive that is at risk of cleavage by intestinal enzymes, from becoming susceptible to intestinal protease cleavage. The pH modulator lowers the pH of the intestinal fluid immediately surrounding the bioactive molecule, reversibly inhibiting the enzymatic cleavage processes of the protease population.

The second protective mechanism of a bioactive pharmaceutical's translocation across the intestinal mucosa is long chain fatty acid coupled translocation. This mechanism requires fat hydrolysis and subsequent component absorption across the apical membrane of an intestinal enterocyte. Upon entry into the gastrointestinal tract, fats are emulsified by bile salts and cleaved at the 1 and 3 sites of the triglyceride molecule, liberating two free fatty acids and a molecule of 2-monoacylglycerol, by pancreatic lipase present in large quantities upon meal consumption. The free fatty acid and 2-monoacylglycerol may then be absorbed from the intestinal lumen into polarized enterocytes lining the small intestine. Although simple diffusion of these molecules is known to occur on a limited scale, a considerable fraction of free fatty acid enters the enterocyte via a fatty acid transporter protein present on the apical brush-border membrane.

Further, the 2-monoacylglycerol is thought to interact with a long chain monoacylglycerol transporter through a related protein-mediated process for transport across the apical membrane. The transport of both molecules is mediated by a single type of free fatty acid transport protein in a competitive manner. Following enterocyte absorption of free fatty acids and monoacylglyceride, these fat hydrolysis products are transported to the endoplasmic reticulum, where typically they are re-incorporated into triglyceride before shuttling through the golgi apparatus. The newly re-incorporated triglyceride is packaged, through processing in these two organelles, with lipoproteins, cholesterol and other lipids into chylomicrons. Upon extrusion of the chylomicrons from the golgi apparatus an exocytotic vesicle is formed and transported to the basolateral aspect of the enterocyte. The vesicles then undergo exocytosis by fusing with the plasma membrane, dumping the chylomicrons into the extracellular matrix. Instead of being absorbed directly into capillary blood, the chylomicron particles are transported through the lymphatic system for disassembly and rapid delivery to the blood.

The oral formulations of the present invention may also be utilized for long chain fatty acid coupled translocation to enhance a molecule's bioavailability. A non-traditional enhanced “piggy-back” translocation manipulates long-chain fatty acid transporters to carry the bioactive across the bi-layer by loosely conjuncting a molecule of fatty acid to the molecule. The association of two molecules is through attractive forces only, not covalent bonding. The result is an enhancement of the bioactive's bioavailability through “piggy-back” translocation.

Alternatively, non-traditional coupled translocation with a fatty acid molecule may be covalently linked to the bioactive. For example, a fatty acid may be covalently linked directly to the amino terminus of the Ceftriaxone molecule through a “peptide” linkage. See FIG. 4 whereby Ceftriaxone is coupled with a fatty acid, such as linoleic acid 40. Ceftriaxone is transported directly by fatty acid molecules as it is transported by its corresponding enterocyte carrier. Initially, the absorption is expected to be identical to the transport process for free fatty acid translocation. The coupled bioactive-fatty acid complex may then ultimately be transported through the endoplasmic reticulum and golgi apparatus before excretion to the circulation. Alternatively, the complex may be directly absorbed into the blood.

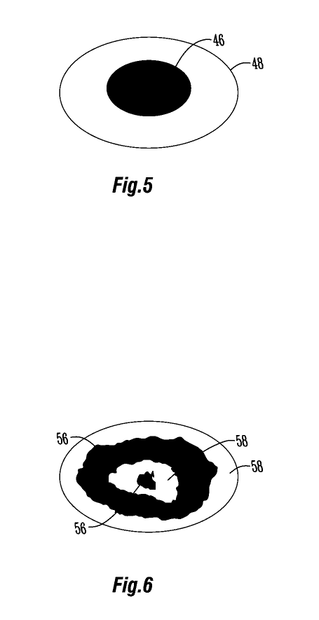

The novel oral formulation of the present invention may be generated in various configurations of size and shape. Although a spherical or oval shaped formulation is referred to most often, it should be understood that numerous variations and modifications may be made to the oral formulation while remaining within the spirit and scope of the invention. FIG. 5 shows the simplest form of the oral formulation, showing its two distinct functional regions. The two separate regions or domains have distinct function. The inner core region 46 functions in the release of both drug and absorbance enhancement components important to enterocyte uptake. The inner core region 46 is surrounded by an outer protective membrane region 48. The outer protective membrane region 48 protects the inner core from the gastric environment and delivers the bioactive pharmaceutical intact to the intestinal mucosa.

The inner core region is unlimited in its bioactive delivery (drug active). The exemplar, oral Ceftriaxone molecules, are one preferred embodiment of the present invention wherein the molecule shows preference to enterocyte uptake at the apical absorptive bi-layer. This preference may be either natural or artificial in the sense of the addition of molecules proven to enhance the bioactives measured translocation across the apical membrane. In one aspect or embodiment of the present invention, the inner core region houses an administrative dose of Ceftriaxone equivalent to approximately 1-2000 mg of active antibiotic. In another embodiment of the oral formulation the administrative dose of Ceftriaxone is 40-1000 mg, with a preferred dose of 500 mg. In addition to antimicrobial, the core also houses 1-2 moles of enhancer, such as sodium chloride, a mono-, di-, or tri-saccharine, glycerin, fatty acid, such as linoleic acid, or other enhancing molecules and combinations thereof. Additionally, enhancers may be added in higher mole ratios.

The inner core region may additionally house a modulating agent. For example, an acid may be added as a modulator to lower the local intestinal luminal pH upon formulation disintegration and ultimately the bioactive's release. A variety of acids may be utilized for this purpose. Citric acid is a preferred acid in a range of 0.01-0.057 mol/kgbw. In one embodiment the citric acid is in an amount approximately 0.031 mol/kgbw. A pH modulator provides the benefit of reversible pH inhibition of intestinal luminal enzymatic activity thus preventing Ceftriaxone cleavage prior to absorption. A protease inhibitor may be used as well. Finally the inner core may contain a small amount of emulsifier such as Tween 20. The inner core region as described above is a liquid droplet with a typical viscosity greater than that of liquids such as milk.

The protective shell, commonly referred to as the protective outer membrane region, is made up initially of sodium alginate. The sodium alginate is converted into a gastric insoluble calcium form in an early stage of the formulation's manufacture, forming a calcium alginate polymer. For example, in one embodiment of the present invention's oral formulation, the calcium alginate is present in a mole ratio of three in comparison to that of Ceftriaxone. A small amount of high fructose corn syrup, 1 mole Ceftriaxone bases, and glycerin is added to aide in polymerization and membrane stability. Additional polymerization additives may include xanthum gum, guar gum, agar, sodium alginate, carrageenan, other polymers and polysaccharides, pharmaceutical equivalents, and combinations of the same. The outer membrane has a texture consistent with that of a rubber material and is extremely resistant to gastric disintegration, but undergoes a dissolution process upon entering the small intestine. Additional protective outer membrane region polymers may be utilized by one of ordinary skill in the art; the calcium alginate polymer is one preferred embodiment.

These novel chemical properties of the outer membrane region also allow a time- or controlled- or extended-release formulation for dose delivery for extended periods of time, such as a period of 24 hours. FIG. 6 depicts a basic example of a time-released formulation showing two layers of a bioactive pharmaceutical dosing 56. The controlled release formulation is yet another embodiment of the present invention that is accomplished through a layering of alginate polymer making up the outer membrane region 58 containing divisional bioactive pharmaceutical dosages 56. Alternatively, additional administrative layers of a bioactive may be added for extended control release.

A benefit to the use of calcium alginate polymer is that it consists of entirely natural components. Additionally, the calcium alginate allows the oral formulation to pass through the gastric cavity without any disintegration. However, upon entry into the small intestine, the formulation quickly disintegrates. In one embodiment of the present invention, the outer membrane region disintegrates within approximately from two to eight minutes.

Dissolution assays utilizing simulated gastric fluids and intestinal fluids illustrated 0.00 disintegration after a 1 hour assay with the compositions of the claimed invention in the simulated gastric fluid. Alternatively, the assay in the simulated intestinal fluid resulted in 100% disintegration in 5.3 minutes. The rate of dissolution is due to the cross-linking of the molecular matrix, preventing the gastric acid and enzymes from inducing the disintegration. Upon entry into the intestines, the varying thickness of the outer membrane region's calcium alginate layer impacts the rate of disintegration.

The manufacturing techniques and procedures for the oral formulations of the present invention described herein are adapted for preparation of the oral formulations for use in a variety of manufacturing operations, including large-scale commercial manufacturing operations. The preparation of the outer membrane solution for atomization requires use of a sanitized vessel to combine the outer membrane components, such as sodium alginate, glycerin, and fructose. Sterile water is added and the mixture is agitated until gellation is achieved. Notably, the polymer solution does not require any heating or adjustment of temperature to achieve gellation under the methods of the present invention. This portion of the method can occur at room temperature or even below, achieving polymer gellation under conditions previously unattainable and resulting in manufacturing methods providing various benefits, including to name a few, increased flexibility and decreased maintenance and operating condition regulation.

Once polymer gellation is achieved, the bioactive pharmaceutical and various pharmaceutical excipients are added and agitated. As soon as a homogenized solution is achieved the atomization process begins. Atomization initially requires a clean and sanitized vessel, to combine and agitate sterile deionized water and the calcium chloride powder. Immediately after the agitation of the calcium chloride solution, an atomization pump is loaded with Beadlet solution and connected to the atomizer head to the calcium chloride reservoir. The pump is primed and powered to enable the atomization of the Beadlet solution.

The atomization process takes place at about 25C. and continues until all of the Beadlet solution has been spent. The Atomizer head is connected to enable the droplets to be sprayed directly into the aqueous calcium chloride solution. The droplets become solid collections directly upon contact with the ionized water and calcium chloride solution. This transformation of the droplets to a solid state presents yet another novel feature of the methods for oral formulation of the present invention and provides various manufacturing benefits.

After the atomization of the Beadlet solution, the beads are allowed to cure within the calcium chloride solution for a period of about 30 minutes. At the end of the curing process, the calcium chloride solution is drained by opening the valve at the bottom of the vessel. The beadlets are then twice washed with 100 kg of water and the dried beadlets are drop-loaded from the Atomization vessel into the dryer tumbler and dried.

### EXAMPLES

This invention can be better understood by reference to the following examples, which are intended to be illustrative of embodiments of the invention, but non-limiting in terms of scope of the invention. It will be appreciated by those skilled in the art that other embodiments of the invention may be practiced without departing from the spirit and the scope of the invention, the scope of which is defined by the appended claims.

### Example 1

Three test formulations evaluated the capacity for Ceftriaxone translocation by the sodium-dependent amino acid transportation mechanism described in the detailed description of the invention. The three different formulations have been identified in the Table 3 with the following designations: F1=Non-traditional “naked encapsulation”; F2=Enhancer mediated “naked encapsulation”; and F3=Enhancer mediated assisted modulator “naked encapsulation.” The formulation process was sequentially identical for each formulation. The sole variant was the ingredient compositions of the inner core region. Table 3 provides each of the formulations. Alternatively, the outer membrane region of the Ceftriaxone formulation is both considered and prepared separately from the inner core region. The procedures used for the preparation of the described formulations were conducted under research settings, however, they may be manufactured on an industrial scale.

TABLE 3

| | Outer Membrane | |
|---|---|---|
| Capsule | mol/1 L | Inner Core |
| F1 | CaAlginate 0.17 | Ceftriaxone |
| | Glycerin 0.043 | 0.00002-0.002 mol |
| | Fructose 0.1 | Tween 20* |
| | Water 55.0 | (*minimal amount used as |
| | | emulsifier) |
| F2 | CaAlginate 0.17 | Ceftriaxone |
| | Glycerin 0.043 | 0.00002-0.002 mol |
| | Fructose 0.1 | Tween 20* |
| | Water 55.0 | NaCl |
| | | 0.00002-0.002 mol |
| F3 | CaAlginate 0.17 | Ceftriaxone |
| | Glycerin 0.043 | 0.00002-0.002 mol |
| | Fructose 0.1 | Tween 20* |
| | Water 55.0 | NaCl |
| | | 0.00002-0.002 mol |
| | | Citric Acid |
| | | 0.031 mol/kgbw |

### Example 2

Two test formulations evaluated the capacity for Ceftriaxone translocation by the fatty acid translocation. The first, non-traditional enhanced “piggy-back” translocation, designated as F4, utilizes a molecule of linoleic acid as enhancer for translocation across the enterocyte membrane. The second, non-traditional coupled translocation, designated as F5, utilizes a molecule of linoleic acid covalently linked to the amino terminus of the Ceftriaxone molecule. It should be understood that any of a number of fatty acid molecules could be used here with comparable enhancement.

The formulation process was sequentially identical for both formulations, with the sole variant being the ingredient compositions of the inner core region. The formulation is prepared in accordance with the description contained in the various examples, differing only in its inner core components. Table 4 provides each of the formulations.

TABLE 4

| | Outer Membrane | |
|---|---|---|
| Capsule | mol/1 L | Inner Core |
| F4 | CaAlginate 0.17 | Ceftriaxone |
| | Glycerin 0.043 | 0.00002-0.002 mol |
| | Fructose 0.1 | Linoleic Acid |
| | Water 55.0 | 0.2 mol/L |
| F5 | CaAlginate 0.17 | Ceftriaxone |
| | Glycerin 0.043 | 0.00002-0.002 mol |
| | Fructose 0.1 | Linoleic Acid |
| | Water 55.0 | 0.2 mol/L |

### Example 3

A formulation procedure for preparing the outer membrane region includes preparing an appropriate amount of stock outer membrane region solution. For example, the outer membrane region of one dosage consists of approximately 1.0 mL of outer membrane region stock solution. The stock solution is prepared using cold water for ease of homogeneity. Once thoroughly mixed, the solution is heated at 100 degrees Celsius for a period of 1-5 minutes. At the end of the heating period, 1.0 mL of hot stock outer membrane region solution is quickly poured into a specialized formulation mold and immediately submerged into calcium chloride solution. Upon submersion into aqueous calcium chloride, a sodium ion from the alginate molecule quickly exchanges with a calcium ion from the added calcium chloride solution. The ionic change occurs instantaneous and results in the generation of a gastric resistant alginate biopolymer which is immediately impregnated with inner core solution.

### Example 4

A formulation procedure for preparing the inner core region includes emulsifying the bioactive, enhancer and modulator in an emulsifier, such as Tween 20, and vortexed for a period of 60 seconds prior to microinjection into the lumen of the outer membrane. Inner core microinjection proceeds after the submersion of the outer membrane into calcium chloride solution, about 30-60 seconds. After impregnation, the formulations are re-submerged into calcium chloride solution and allowed to stand to appropriate loss of membranous moisture, about 3-10 minutes. However, the oral formulations may be removed from the calcium chloride solution as early as 20 seconds after the second submersion process, depending upon the desired percent moisture content.

The oral formulation may also be coated with additional functional or cosmetic membranes, such as a carrageenan outer shell, and dried according to desire.

### Example 5

Oral Ceftriaxone formulations were administered to rabbit test subjects through a formulation delivery tube to permit oral administration, illustrative of the same absorption processes as the human stomach and intestines. Rabbits were dosed at a Ceftriaxone load of 40-80 mg depending upon body weight. Rabbits were dosed with all five oral Ceftriaxone formulations and absorption data collected by HPLC analysis of plasma Ceftriaxone concentrations from the heparinized rabbit plasma specimen. Blood draws were collected at pre-determined intervals of time, spun down to separate serum, and serum was removed and frozen prior to delivery to Clinical Pathology Laboratories in Oklahoma City, Okla.

The following data table shows the draw times and plasma concentrations after both oral administration and IV administration of Ceftriaxone sodium.

TABLE 5
Study Sample Concentration (mcg/mL)

| Animal | 0.00 | 0.5 | 1.0 | 2.0 | 4.0 | 6.0 |
|---|---|---|---|---|---|---|
| Number | Hours | Hours | Hours | Hours | Hours | Hours |
| 001 | BLQ | 31.84 | 21.71 | 14.47 | 5.79 | 4.34 |
| 002 | BLQ | 110.88 | 75.60 | 50.40 | 20.16 | 15.12 |
| 003 | BLQ | 143.15 | 97.60 | 65.07 | 26.03 | 19.52 |
| 004 | BLQ | 35.69 | 24.33 | 16.22 | 6.44 | 4.87 |
| 005 | BLQ | 40.194 | 27.41 | 18.27 | 7.31 | 5.48 |

(Control) Plasma Concentration of Ceftriaxone Over Time After IV
Administration

| | | | | | | |
|---|---|---|---|---|---|---|
| 006 | BLQ | 198.21 | 135.63 | 90.81 | 36.43 | 27.34 |

BLQ = Below Limit of Quantitation (<1.00 mcg/mL)

### Example 6

Dissolution assays for the formulation of gastric resistant formulations for intestinal delivery utilized the following prepared solutions: simulated gastric fluids and simulated intestinal fluids.

The simulated gastric fluid was made pursuant to and with the following techniques, procedures, reagents and equipment. In a 1000 mL flask 2.0 g of sodium chloride and 3.2 g of pepsin were weighed. 7.0 mL of concentrated hydrochloric acid was slowly added and sufficient water to make 1000 mL was added. The pH was checked and adjusted to 1.2 if outside the range of about from 1 to 3.

The simulated intestinal fluid was made pursuant to and with the following techniques, procedures, reagents and equipment. 6.8 g of mono-basic potassium phosphate was dissolved in 250 mL of water and mixed well. Then 77 mL of 0.2N sodium hydroxide and 500 mL of water were added. 10 g of pancreatin was added and mixed. The resultant solution was adjusted with either 0.2 N sodium hydroxide or 0.2N hydrochloric acid to a pH of 6.8, if outside the range of from about 6 to 8. The solution was diluted to 1000 mL with distilled water.

The 1000 mL of gastric fluid was heated to 37C. before the oral dosage formulation of the present invention was placed into the basket of a homogenizer. The apparatus was assembled, loaded with the gastric vessel. The homogenizer was observed at a speed of 1 for an hour, noting the point at which disintegration initiated and completed. At the end of 1 hour, any dose remaining in the basket was transferred into the simulated intestinal fluid, heated to 37C. and observed to noted the initiation and completion of disintegration.

The gastric fluid resulted in 0.00 disintegration after a 1 hour assay with the compositions of the claimed invention, whereas the assay in simulated intestinal fluid resulted in 100% disintegration in 5.3 minutes.

### Example 7

The preparation of the outer membrane solution for atomization utilizes the following techniques, procedures, reagents and equipment. A sanitized 55 gallon stainless steel vessel is tarred and its weight recorded. 6.4 kg of sodium alginate, 0.200 kg of glycerin, 0.050 kg of fructose are added to the vessel. 60 gallons of sterile water are added under agitation and the mixture is promptly agitated at 1200 rpm at 25C. until gellation is achieved. The gellation may take place at without heating the components, at room temperature or below, yielding the same results.

Then 2.5 kg of 98% ceftriaxone sodium and 0.400 kg Tween 20 were added and agitated for 30 minutes before adding 0.056 kg of concentrated CitroBio and mixed for an additional 30 minutes. Once the homogenized solution is achieved the atomization process begins.

Atomization initially requires a clean and sanitized 55 gallon vessel, tarred and recorded. 50 kg of sterile deionized water and 11 kg of calcium chloride powder are added and slowly agitated for 15 minutes. Immediately after the agitation of the calcium chloride solution, the atomization pump is loaded with Beadlet solution and connected to the atomizer head to the calcium chloride reservoir. The pump is primed and powered so the atomization of the Beadlet solution may begin.

The atomization process takes place at 25C. and continues until all of the Beadlet solution has been spent. The Atomizer head should be connected such that the droplets are sprayed directly into the aqueous calcium chloride solution. After the atomization of the Beadlet solution, the beads are allowed to cure within the calcium chloride solution for a period of 30 minutes. At the end of the curing process, the calcium chloride solution is drained by opening the valve at the bottom of the vessel. The beadlets are then twice washed with 100 kg of water. The dried beadlets are drop-loaded from the Atomization vessel into the dryer tumbler and dried for a period of 1 hour at 42C. to a final weight of 6.0 kg.

We claim:

1. An oral bioactive pharmaceutical formulation comprising:
a therapeutically effective amount of a bioactive pharmaceutical contained in an inner core region; and
a protectant outer membrane region surrounding the inner core region, wherein the outer membrane region promotes delivery of the bioactive pharmaceutical directly to intestinal mucosa.

2. The oral formulation of claim 1 wherein the bioactive pharmaceutical is a water-soluble.

3. The oral formulation of claim 1 wherein the bioactive pharmaceutical is a lipid-soluble.

4. The oral formulation of claim 1 wherein the bioactive pharmaceutical is any drug active molecule capable of oral ingestion.

5. The oral formulation of claim 1 wherein the bioactive pharmaceutical is a drug active selected from the group consisting of pharmaceuticals, neutraceuticals, vitamins, minerals, vaccines, RNA, DNA, peptides, peptide mimics and combinations of the same.

6. The oral formulation of claim 1 wherein the inner core region further comprises a therapeutically effective amount of additives promoting intestinal translocation and absorption selected from the group consisting of targeting molecules, protectant molecules, lipid-soluble base mediums, water-soluble base mediums and combinations thereof.

7. The oral formulation of claim 1 wherein the inner core region further comprises a therapeutically effective amount of translocation enhancers for enterocyte uptake.

8. The oral formulation of claim 7 where the translocation enhancers are selected from the group consisting of sodium chloride, mono-saccharine, di-saccharine, tri-saccharine, glycerin, fatty acids, pharmaceutical equivalents and combinations thereof.

9. The oral formulation of claim 8 where the fatty acid is linoleic acid.

10. The oral formulation of claim 6 wherein the absorbance enhancers are present in an amount approximately from 1 to 2 moles.

11. The oral formulation of claim 7 wherein the inner core region further comprises a modulating agent to prevent cleavage of the bioactive pharmaceutical prior to absorption.

12. The oral formulation of claim 11 wherein the modulating agent is selected from the group consisting of an acidic agent, a protease inhibitor and combinations thereof.

13. The oral formulation of claim 12 wherein the acidic agent is citric acid.

14. The oral formulation of claim 13 wherein the citric acid is present in the amount of approximately from 0.01-0.057 mol/kgbw.

15. The oral formulation of claim 14 wherein the citric acid is present in the amount of approximately 0.031 mol/kgbw.

16. The oral formulation of claim 7 wherein the inner core region further comprises an emulsifier.

17. The oral formulation of claim 16 wherein the emulsifier is Tween 20.

18. The oral formulation of claim 1 wherein the outer membrane region comprises a gastric insoluble calcium alginate polymer.

19. The oral formulation of claim 18 wherein the calcium alginate polymer to bioactive pharmaceutical ratio is 3:1.

20. The oral formulation of claim 18 wherein the outer membrane region further comprises polymerization additives selected from the group consisting of high fructose corn syrup, Ceftriaxone bases, glycerin, water, xanthum gum, guar gum, agar, sodium alginate, carrageenan, other pharmaceutically equivalent polymers and polysaccharides, and combinations thereof.

21. The oral formulation of claim 20 wherein the outer membrane further comprises additional functional or cosmetic membranes.

22. The oral formulation of claim 21 wherein the additional membrane is a carrageenan outer shell.

23. The oral formulation of claim 18 wherein the insoluble calcium alginate polymer is alternately layered with the bioactive pharmaceutical to provide a controlled release formulation.

24. A method of formulating a gastric resistant oral formulation for intestinal delivery comprising:
emulsifying a bioactive pharmaceutical, an enhancer and a modulator in an emulsifier to form an inner core region;
forming an outer membrane region comprising droplets of calcium alginate;
microinjecting the inner core region into the outer membrane region;
adding polymerization additives to the calcium alginate for membrane stability;
submerging the oral formulation into calcium chloride solution; and
drying the oral formulation.

25. The method of claim 24 wherein forming the outer membrane region further comprises agitating members selected from the group consisting of sodium alginate, glycerin, fructose, pharmaceutical equivalents and combinations of the same until polymer gellation occurs.

26. The method of claim 24 wherein polymer gellation occurs below or above room temperature.

27. The method of claim 24 further comprising atomizing the outer membrane region to droplets prior to submerging the droplets into the calcium chloride solution.

28. The method of claim 25 wherein the droplets transition into solid collections of the outer membrane region upon contacting the calcium chloride solution.

29. The method of claim 28 further comprising curing the droplets within the calcium chloride solution for a period of about 30 minutes.

30. The method of claim 29 further comprising washing the droplets with water before drying the oral formulation.

31. The method of claim 24 further comprising the step of adding additional functional or cosmetic membranes to the oral formulation.

32. The method of claim 24 further comprising the step of layering additional inner core regions and outer membrane regions to provide a controlled release formulation.

33. A method of mammalian administration of an oral formulation resistant to
gastric disintegration, comprising:
administering an oral formulation to a patient in need thereof;
causing the oral formulation to enter the gastric cavity;
bypassing gastric disintegration of the oral formulation;
undergoing dissolution of an outer membrane region of the oral formulation upon encountering small intestine digestive enzymes;
releasing a therapeutically effective amount of solubilized bioactive pharmaceutical from an inner core region;
translocating the bioactive pharmaceutical across intestinal mucosa; and
absorbing the bioactive pharmaceutical into the blood.
